(19) United States
(12) Patent Application Publication
(10) Pub. No.: US 2022/0169617 A1
(43) Pub. Date: Jun. 2, 2022

(54) CONTINUOUS PROCESS FOR PRODUCING BENZIMIDAZOLE DERIVATIVES

(71) Applicants: Helsinn Healthcare SA, Pazzallo-Lugano (CH); HELSINN ADVANCED SYNTHESIS SA, Biasca (CH)

(72) Inventors: Loïc JOST, Fribourg (CH); Christophe ALLEMANN, Fribourg (CH); Luca FADINI, Biasca (CH); Christophe VIGLIOTTA, Fribourg (CH)

(73) Assignee: HELSINN HEALTHCARE SA, Pazzallo-Lugano (CN)

(21) Appl. No.: 17/438,587

(22) PCT Filed: Mar. 12, 2020
(86) PCT No.: PCT/IB2020/000181

§ 371 Date: Sep. 13, 2021

Related U.S. Application Data
(60) Provisional application No. 62/818,627, filed on Mar. 14, 2019.
(60) Provisional application No. 62/895,349, filed on Sep. 3, 2019.

(51) Int. Cl. C07D 233/61

(52) U.S. Cl. CPC C07D 233/61

(57) ABSTRACT

Disclosed are continuous processes for producing benzimidazole derivative Compound I comprising reduction of Compound 2 and reaction with valeraldehyde and conditions and apparatuses for the same.

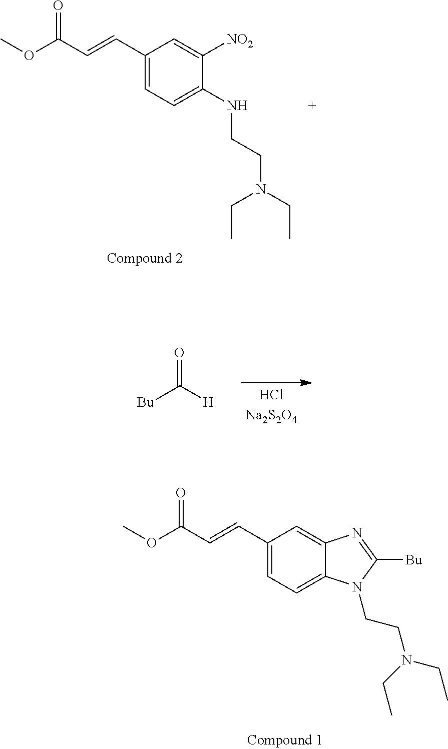

This is application claims the benefit of U.S. Provisional Application No. 62/818,627, filed Mar. 14, 2019, and U.S. Provisional Application No. 62/895,349, filed Sep. 3, 2019, all of which are incorporated herein by reference.

The present disclosure relates to continuous processes for producing benzimidazole derivative Compound 1 comprising reduction of Compound 2 and reaction with valeraldehyde and conditions and apparatuses for the same. Scheme 1 depicts the synthetic process.

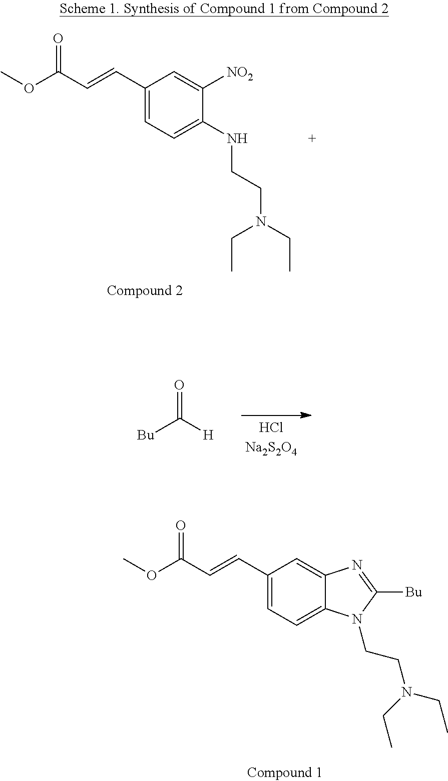

As used herein, Compound 1 refers to methyl (E)-3-(2-butyl-1-((diethylamino)-methyl)-1H-benzoimidazol-5-yl)acrylate (also called “06-PRAN”). The structure of Compound 1 is shown above.

As used herein, Compound 2 refers to methyl (E)-3-(4-((2(diethylamino)ethyl)-amino)-3-nitrophenyl)acrylate (also called “04-PRAN”). The structure of Compound 2 is shown above.

As shown above, the continuous process comprises reacting Compound 2 to reduce its nitro group and cyclizing the resulting reduced product (an amine intermediate) with valeraldehyde to produce Compound 1.

The inventors' initial investigations of the continuous process that form a basis of the presently claimed continuous process are detailed in priority applications U.S. Provisional Application No. 62/818,627, filed Mar. 14, 2019, and U.S. Provisional Application No. 62/895,349, filed Sep. 3, 2019, the entireties of which are incorporated herein by reference.

In summary, initial investigations leading to the continuous processes disclosed herein were performed in a mesoreactor-based continuous system. It was discovered that Compound 2, which has a limited solubility in methanol, crystallized inside the connectors and clogged them. To prevent crystallization issues, the solution of Compound 2 was heated and maintained at least at 45° C. between its flask and the coiled reactor, which was heated at 80° C. 10% (V/V) tetrahydrofuran (THF) was also added to Compound 2. To prevent backflow issues, the choice of pump was made with consideration, and suitable pumps included Vapourtec's peristaltic pumps, which are designed to work under pressure in a continuous system.

Each of the two reactions in the synthetic step were then studied separately. During the study of the reduction of Compound 2 with sodium dithionite, the inventors determined that clogging issues could be minimized if this reaction was carried out at 80° C. under atmospheric pressure. Additionally, by increasing the ratio of water to sodium dithionite flow rates, the inventors discovered that the reagent equivalents could be reduced from 8.6 to 2.4 eq. while still achieving a complete conversion. Nonetheless at 2.4 equivalents, the dilution of the amine intermediate of Compound 2 was 0.006 mmol/g of reaction mixture. By using 3.7 eq., the final concentration of amine intermediate of Compound 2 was increased to 0.015 mmol/g. It was discovered that the use of more concentrated conditions improved the costs and the productivity of the reduction reaction.

During the study of the formation of the benzimidazole ring from the amine intermediate of Compound 2, the inventors discovered that a continuous process for this reaction using the same conditions as for a batch process did not result in any conversion. The inventors also explored the effects of changes to temperature, pressure, residence time, starting material concentration, proportions of valeraldehyde and hydrochloric acid.

An increase in yield under the conditions tested was achieved using 8.4 eq. and 15 eq. of valeraldehyde and hydrochloric acid respectively. Once the reactions had been investigated separately, the synthesis of Compound 1 from Compound 2 was investigated. The investors discovered that a continuous process should comprise a system and use conditions that avoids clogging by insoluble particles of sodium dithionite and/or by a solid by-product of the reaction. The inventors discovered a continuous process for the synthesis of Compound 1 from Compound 2 wherein hydrochloric acid is added to the system before a back pressure regulator (BPR) in the apparatus, to degrade insoluble particles of sodium dithionite. In addition, the inventors discovered that, if the apparatus for the continuous process were designed such that the length of the tube between the end of the second reactor (R02) and the entrance of the flask collecting Compound 1 (B06) was sufficiently short and such that valeraldehyde was introduced after the BPR, the by-product precipitation occurred inside the Compound 1 flask, no longer in the tube, such that the overall synthesis was able to be carried out in a continuous mode. See FIGS. 1-5.

The inventors continued their investigations of continuous processes for the synthesis of Compound 1 from Compound 2, which resulted in discoveries that led to various changes, including shortening of the tube connecting the dithionite flask to its pump (e.g., FIG. 8), inversion of pumps and use of a cartridge as a packed bed reactor for sodium dithionite (e.g., FIG. 9).

Accordingly, as disclosed herein, the inventors discovered novel continuous processes for performing the reaction of Compound 2 to form Compound 1.

### BRIEF DESCRIPTION OF THE FIGURES

FIG. 1 illustrates symbols used in the apparatuses of the disclosure.

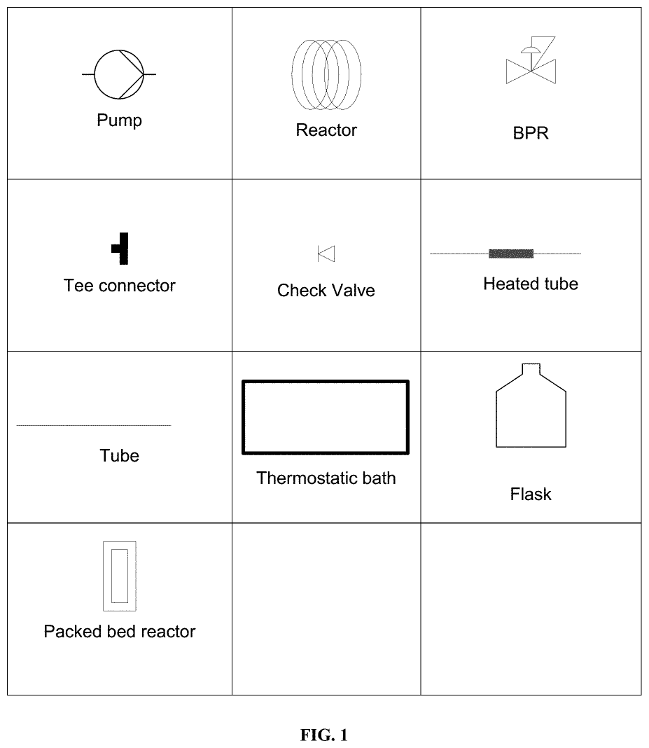

FIG. 2 depicts an apparatus and conditions for the disclosed continuous process.

FIG. 3 depicts an apparatus and conditions for the disclosed continuous process, wherein the value given for reactors corresponds to the length of the reactors and the numbers in black represent the length of the tube between two connectors.

FIG. 4 depicts another apparatus and conditions for the disclosed continuous process.

FIG. 5 depicts another apparatus and conditions for the disclosed continuous process.

FIG. 6 depicts another apparatus and conditions for the disclosed continuous process.

FIG. 7 depicts another apparatus and conditions for the disclosed continuous process.

FIG. 8 depicts another apparatus and conditions for the disclosed continuous process using a shortened tube between the dithionite flask and pump.

FIG. 9 depicts another apparatus and conditions for the disclosed continuous process, wherein a cartridge is used as a packed bed reactor for dithionite.

FIG. 10 depicts a UPLC trace for Compound 1 after subtraction of blank.

FIG. 11 depicts mass spectroscopy traces for Compound 1 and byproduct.

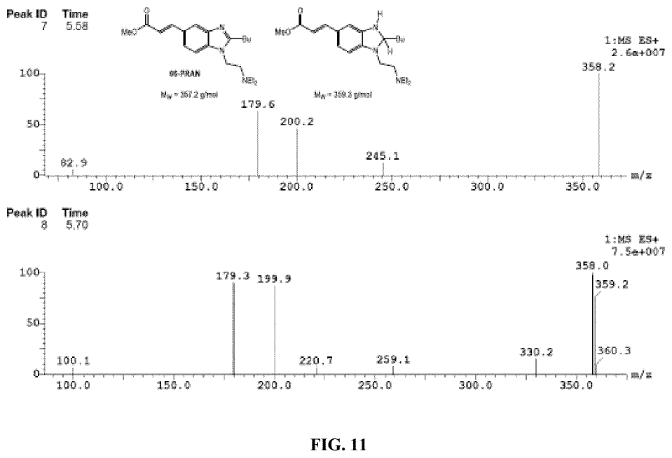

FIG. 12 depicts ¹H NMR spectrum for Compound 1.

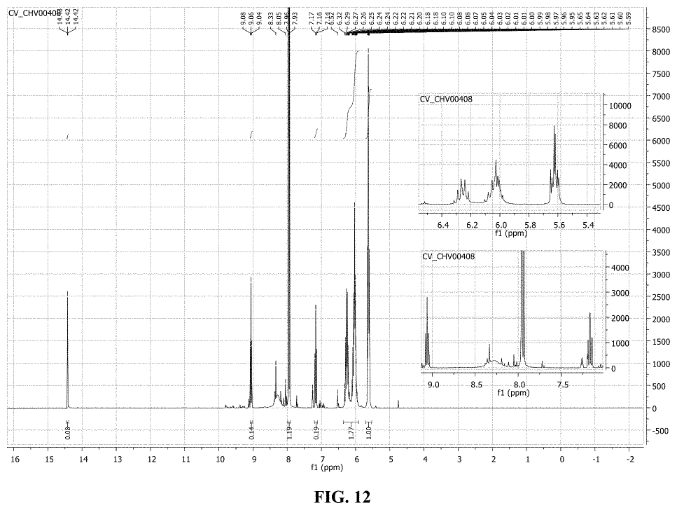

Disclosed herein are continuous processes for producing Compound 1 from Compound 2 and valeraldehyde.

In some embodiments, disclosed herein is a continuous process comprising reacting Compound 2 to reduce its nitro group and cyclizing the product with valeraldehyde to produce Compound 1,

wherein a composition comprising Compound 2 and optionally at least one solvent is continuously passed through a packed bed reactor comprising sodium dithionite,

wherein the resulting composition is continuously transferred to an continuous process reactor,

wherein the composition comprising the reaction product of sodium dithionite and Compound 2 is combined with valeraldehyde and passed through a back pressure regulator,

wherein hydrochloric acid is combined with the composition comprising the reaction product of sodium dithionite, Compound 2, and valeraldehyde, and

wherein the resulting composition is continuously transferred to an additional continuous process reactor, and

wherein Compound 1 is continuously produced.

In some embodiments, the continuous process reactor and the additional continuous process reactor are in a thermostatic bath. In some embodiments, the thermostatic bath is heated to 80° C.

In some embodiments, the composition comprising Compound 2 further comprises water. In some embodiments, the composition comprising Compound 2 is heated. In some embodiments, the composition comprising sodium dithionite is heated.

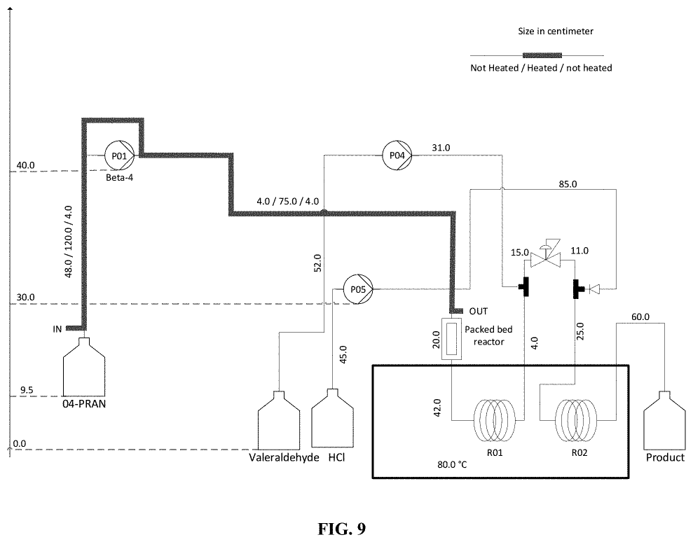

In some embodiments, the apparatus used for the continuous process is substantially the same as shown in FIG. 9.

In some embodiments, disclosed herein is a continuous process comprising reacting Compound 2 to reduce its nitro group and cyclizing the product with valeraldehyde to produce Compound 1,

wherein a composition comprising Compound 2 and optionally at least one solvent is continuously combined with a composition comprising sodium dithionite and optionally at least one solvent,

wherein the composition comprising the reaction product of sodium dithionite and Compound 2 is combined with water,

wherein the resulting composition is continuously transferred to an continuous process reactor,

wherein the composition comprising the reaction product of sodium dithionite, Compound 2, and water is combined with valeraldehyde and passed through a back pressure regulator,

wherein hydrochloric acid is combined with the composition comprising the reaction product of sodium dithionite, Compound 2, and valeraldehyde, and

wherein the resulting composition is continuously transferred to an additional continuous process reactor, and

wherein Compound 1 is continuously produced.

In some embodiments, the continuous process reactor and the additional continuous process reactor are in a thermostatic bath. In some embodiments, the thermostatic bath is heated to 80° C. In some embodiments, the thermostatic bath further comprises a heater through which water continuously passes.

In some embodiments, the composition comprising Compound 2 is heated. In some embodiments, the composition comprising sodium dithionite is heated. In some embodiments, the water combined with the composition comprising the reaction product of sodium dithionite and Compound 2 is heated. In some embodiments, the water is heated to 80° C.

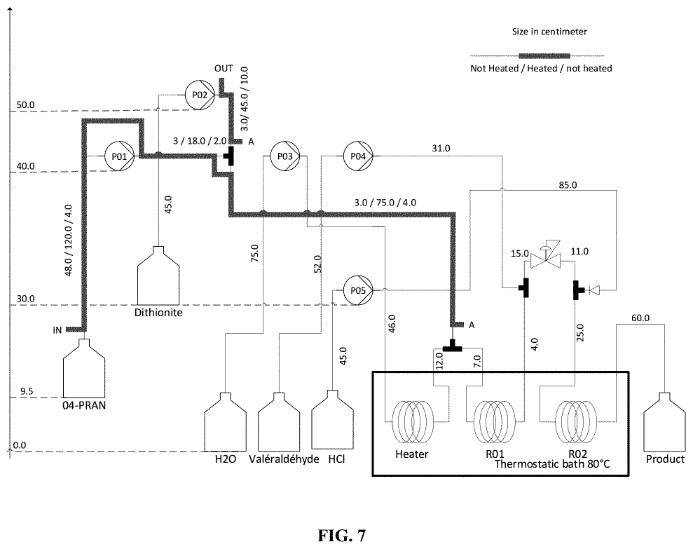

In some embodiments, the apparatus used for the continuous process is substantially the same as shown in at least one Figure chosen from FIGS. 7, 8, and 9.

In some embodiments, disclosed herein is a continuous process comprising reacting Compound 2 to reduce its nitro group and cyclizing the product with valeraldehyde to produce Compound 1,

wherein a composition comprising Compound 2 and optionally at least one solvent is continuously transferred to a continuous process reactor,

wherein sodium dithionite is continuously combined with the composition comprising Compound 2,

wherein the composition comprising the reaction product of sodium dithionite and Compound 2 is combined with hydrochloric acid,

wherein the composition comprising the reaction product of sodium dithionite and Compound 2 is combined with hydrochloric acid, followed by passage through a back pressure regulator,

wherein valeraldehyde is combined with the composition comprising the reaction product of sodium dithionite, Compound 2, and hydrochloric acid, and the resulting composition is continuously transferred to an additional continuous process reactor, and

wherein Compound 1 is continuously produced.

In some embodiments, the continuous process reactor and the additional continuous process reactor are in a thermostatic bath. In some embodiments, the thermostatic bath is heated to 80° C. In some embodiments, the thermostatic bath further comprises a heater through which water continuously passes.

In some embodiments, the pressure of the continuous process reactor is 4.0 bar. In some embodiments, the pressure of the additional continuous process reactor is atmospheric pressure.

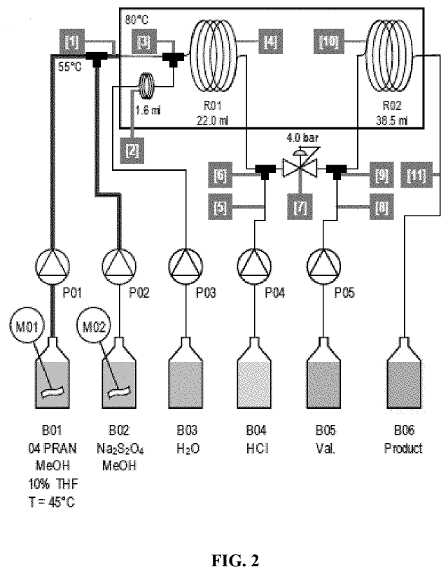

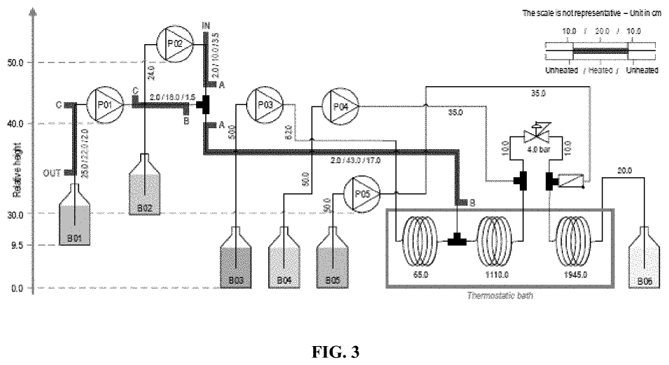

In some embodiments, the apparatus used for the continuous process is substantially the same as shown in at least one Figure chosen from FIGS. 2 and 3.

In some embodiments, disclosed herein is a continuous process comprising reacting Compound 2 to reduce its nitro group and cyclizing the product with valeraldehyde to produce Compound 1,

wherein a composition comprising Compound 2 and optionally at least one solvent is continuously combined with a composition comprising sodium dithionite and optionally at least one solvent,

wherein water is added to the composition comprising the reaction product of sodium dithionite and Compound 2,

wherein the composition comprising water and the reaction product of sodium dithionite and Compound 2 is continuously transferred to a continuous process reactor,

wherein the composition comprising water and the reaction product of sodium dithionite and Compound 2 is combined with valeraldehyde,

wherein hydrochloric acid is combined with the composition comprising the reaction product of sodium dithionite, Compound 2, and valeraldehyde,

wherein the resulting composition is continuously transferred to an additional continuous process reactor, and

wherein Compound 1 is continuously produced.

In some embodiments, the composition comprising Compound 2 further comprises at least one solvent. In some embodiments, the at least one solvent is chosen from methanol, tetrahydrofuran, and water. In some embodiments, the at least one solvent is methanol and 10% tetrahydrofuran. In some embodiments, the composition comprising Compound 2 further comprising at least one solvent is heated to 45° C.

In some embodiments, the composition comprising sodium dithionite further comprises at least one solvent. In some embodiments, the at least one solvent is chosen from methanol and water. In some embodiments, the at least one solvent is methanol.

In some embodiments, the composition comprising Compound 2 and optionally at least one solvent is heated. In some embodiments, the composition comprising Compound 2 and optionally at least one solvent is heated to 55° C. In some embodiments, the composition comprising sodium dithionite and optionally at least one solvent is heated.

In some embodiments, the continuous process reactor and the additional continuous process reactor are in a thermostatic bath. In some embodiments, the thermostatic bath is heated to 80° C. In some embodiments, the thermostatic bath further comprises a heater through which water continuously passes.

In some embodiments, after passing through the additional continuous process reactor, the composition is continuously passed through a cooling loop and/or through a back pressure regulator.

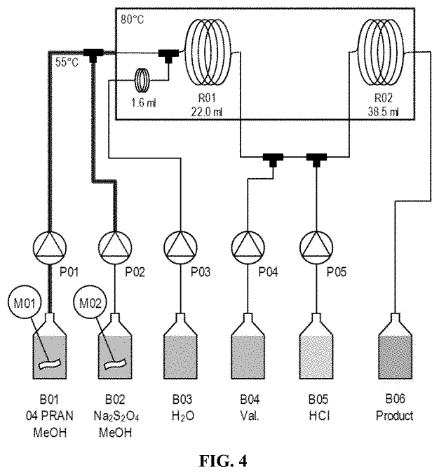

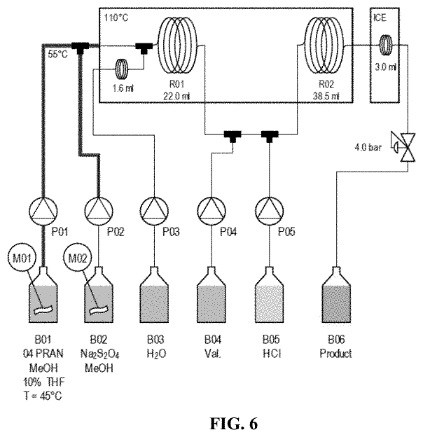

In some embodiments, the apparatus used for the continuous process is substantially the same as shown in at least one Figure chosen from FIGS. 4, 5, and 6.

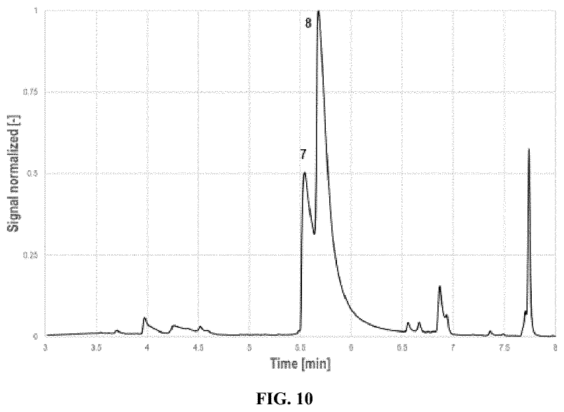

In some embodiments, a composition comprising Compound 2 and optionally at least one solvent is continuously transferred to a continuous process reactor (e.g., RO1 in FIGS. 2-10) by a pump (e.g., PO1 n FIGS. 2-10). In some embodiments, the pump is a membrane pump. In some embodiments, the membrane pump is chosen from a SIMDOS 02 RS+ membrane pump by KNF or a pump similar thereto having a pressure range up to 6 baru and flow rate range of 0.03 ml/min to 20 ml/min., a Beta 4 membrane pump by Prominent or a pump similar thereto having a pressure range up to 8 baru and flow rate range of 0.76 ml/min to 30 ml/min., and a Gamma 4 membrane pump by Prominent or a pump similar thereto having a pressure range up to 10 baru and flow rate range of 0.8 ml/min to 49 ml/min. In some embodiments, the composition comprising Compound 2 and optionally at least one solvent is continuously transferred to a continuous process reactor at a temperature greater than room temperature, such as, for example, 25° C., 35° C., 45° C., 55° C., 65° C., 75° C., 80° C., 85° C.

In some embodiments, sodium dithionite is combined with the composition comprising Compound 2 prior to transfer to a continuous process reactor, such as reactor RO1. In some embodiments, the combination of sodium dithionite and the composition comprising Compound 2 is transferred to the continuous process reactor at a temperature greater than room temperature, such as, for example, 25° C., 35° C., 45° C., 55° C., 65° C., 75° C., 80° C., 85° C.

In some embodiments, the disclosed continuous process further comprises adding at least one solvent to the sodium dithionite, the composition comprising Compound 2, and/or the combination of sodium dithionite and the composition comprising Compound 2. In some embodiments, the at least one solvent is chosen from organic solvents and water. In some embodiments, the at least one solvent is chosen from water, methanol, and tetrahydrofuran. In some embodiments, the at least one solvent is water. In some embodiments, the at least one solvent is added to the sodium dithionite. In some embodiments, the at least one solvent is added to the composition comprising Compound 2. In some embodiments, the at least one solvent is added to the combination of sodium dithionite and the composition comprising Compound 2. In some embodiments, the at least one solvent is added to the combination of sodium dithionite and the composition comprising Compound 2 prior to transfer to a continuous process reactor, such as reactor RO1.

In some embodiments, the sodium dithionite and the composition comprising Compound 2 are reacted in the continuous process reactor, such as reactor RO1, until the reduction of the nitro group of Compound 2 is sufficiently complete.

In some embodiments, the combination formed from the addition of hydrochloric acid to the reaction product of sodium dithionite and Compound 2 is continuously flowed through a back pressure regulator.

In some embodiments, the additional continuous process reactor to which the combination formed from valeraldehyde and the composition comprising the reaction product of sodium dithionite, Compound 2, and hydrochloric acid is continuously transferred is the second continuous process reactor, such as reactor RO2 in FIGS. 2-10.

### EXAMPLES

### Materials and Methods

### 1. Materials

### 1.1. Tube and Other Elements Related to a Flow Process

Tubes used for the different parts of the installation—including the reactor—were made of perfluoroakloxy (PFA), a fluoropolymer presenting an excellent chemical compatibility with the chemicals used for this synthesis. These tubes have an i.d. of 1/16 inch (1.588 mm) and an o.d. of ⅛ inch (3.175 mm). They are equipped with 1/4-28 UNF flat polypropylene (PP) flanged fitting.

Tee connectors and unions are made of polyetrafluoroethylene (PTFE).

The tubes used to create the double jacket for PFA tubes had an i.d. of 5 mm and an o.d. of 8 mm, they were formulated in polyvinyl chloride (PVC).

### 1.2. Thermostatic Baths

The characteristics of the two thermostatic baths are summarised in Table 1.

TABLE 1
Characteristics of the thermostatic baths

| | | |
|---|---|---|
| Manufacturer | Julabo | Haake |
| Model(s) | MV-4 | N2-B or F3-S |
| Temperature range | 20→200° C. | 45→250° C. |
| Purpose | Heat the reactor(s) and any | Heat the jacket |
| | water heater | |

### 1.3. Back Pressure Regulator

A back pressure regulator is a device used to increase the pressure in the reactor located before it. It is composed of a chamber where a membrane compresses a tube where liquid passes by. When a set pressure is given on the membrane chamber, the liquid pressure going through it must match the membrane pressure to go across the regulator.

The device has two operating mode: “Static” and “Mobile.” In case of “Static” use, the device is connected directly to the pressurised air network and the air flow is given continuously. In case of “Mobile” use, the device is set to the desired pressure using the network, the isolation valve on the BPR is closed, and then the device is connected to the installation. For the experiments herein, the “static” mode was used. Characteristics of the device can be found in Table 2.

TABLE 2
Characteristics of back pressure regulator

| | |
|---|---|
| Manufacturer | Zaiput |
| Model | BPR-10 |
| Pressure range | 0→20 baru |
| Flow rate range | 0.05→20 ml/min |
| Maximum temperature | 130° C. |

### 1.4. Membrane Pumps

| Manufacturer | KNF |
|---|---|
| Model | SIMDOS 02 RS+ |
| Pressure range | 0→6.0 baru |
| Flow rate range | 0.03→20 ml/min |
| Internal code | P053 |
| Misc. | Equipped with a 70 μm filter to |
| | protect from particles |
| Manufacturer | Prominent |
| Model | Beta 4 |
| Pressure range | 0→8 baru |
| Flow rate range | 0.076→30 ml/min |
| Manufacturer | Prominent |
| Model | gamma 4 |
| Pressure range | 0→10 baru |
| Flow rate range | 0.8→48.60 ml/min |

### 1.5. Peristaltic Pumps

| Manufacturer: | Ismatec |
|---|---|
| Model: | REGLO Analog MS4/12 |
| | (ISM796) |
| Pressure range: | 0 . . . 1 bar |
| Flow rate ranges: | 0.002 . . . 24 ml/min |
| Working temperatue: | 5 . . . 40° C. |
| HEIA-FR Code: | P072 & P073 |
| Other features: | Used with Tygon MHLL tube |
| | (i.d. = 2.79 mm) |
| Manufacturer: | Vapourtec |
| Model: | SF-10 |
| Pressure range: | 0 . . . 10 bar |
| Flow rate range: | 0.02 . . . 10 ml/min |
| Working temperature: | Not specified |
| HEIA-FR Code: | P074 & P075 |
| Other features: | Used with the blue tube |

1.6. Rotary piston pumps

| | |
|---|---|
| Manufacturer | Ismatec |
| Model | REGLO-CPF Digital |
| Pressure range | 0→6.9 baru |
| Flow rate range | 0.1→45 ml/min |
| Internal code | P043 and P044 |

### 2. Methods

Samples were analyzed using UHPLC and UPLC-MS.

### 2.1. UHPLC Method

| Device | Vanquish Flex (Serial Number 8302616) | | |
|---|---|---|---|
| Column | Waters UPLC CSH C18 1.7 μm × | | |
| | 2.1 mm × 100 mm | | |
| Flow rate | 0.6 ml/min | | |
| Wavelength and detector | UV at 280 nm | | |
| Column temperature | 35° C. | | |
| Sampler temperature | 25° C. | | |
| Volume of injection | 1 μl | | |
| Washing solution | MeOH/H2O 30:70 | | |
| Washing procedure | Mode: both/20 s at 50 μl/s | | |
| Mobile phase | [A]: H₂O + 0.1% TFA | | |
| | [B]: ACN + 0.1% TFA | | |
| Program | Time | A | B |
| | 0.0 | 97 | 03 |
| | 2.0 | 97 | 03 |
| | 5.5 | 85 | 15 |
| | 7.6 | 63 | 37 |
| | 12.0 | 05 | 95 |
| | 13.5 | 05 | 95 |
| | 13.6 | 97 | 03 |
| | 20.0 | 97 | 03 |

| Retention time for products | Product | Retention time (min) |
|---|---|---|
| | Compound 2 | 8.8 |
| | Compound 1 | 7.9 |

### 2.2. Pump Calibration

Pumps are calibrated by weighing an empty weighed flask positioned at the output of the pump. The mixture/chemical is pumped during a certain time at the highest possible flow rate. Then, the flask is weighed in order to determine the amount of mixture pumped.

Using both mass and the time, the mass flow in [g/min] can be determined. The operations are repeated at different flow rates and a linear regression between the input [ml/min] and the mass flow [g/min] is performed.

### Example 1

### A. Operating Procedures for Example 1

### Preparation of Solutions

Compound 2 is introduced into the flask B01, then methanol is added to obtain a solution of 15 g/l 10% (V/V) of THF are added to flask B01. The mixture is stirred at 400 rpm and heated at 45° C., e.g., using a heated stirring plate.

Where an apparatus comprising a cartridge is used, water is added in the B01 flask to obtain a solution where water represents 140 equivalents of Compound 2.

A B02 flask is first washed with acetone and then dried over pressurized air. Methanol is then introduced into the flask and the solvent is flushed with argon (the bottle is topped with a balloon filled with argon). Finally, sodium dithionite is introduced into the flask. Another flush can be performed. Quantities sufficient to obtain a 36 g/l suspension of sodium dithionite in methanol are used.

Water, valeraldehyde and hydrochloric acid are added to their respective flasks. Flasks are all equipped with a ventilation valve and a pumping tube, except the dithionite flask B02 which is equipped with an argon balloon instead of a ventilation valve. The B01 flask was also equipped with a thermometer.

Where an apparatus comprising a cartridge is used, the cartridge is cleaned with water and then acetone then dried over pressurised air being careful not to expel the filters from the cartridge. The sodium dithionite is then inserted in the desired mass and packed using the cartridge wheel.

### Start-Up of the Installation

Once the different parts of the installation are connected, thermostatic baths are switched on at their respective desired temperatures (jacket and reactor). The tube between the P03 pump and the Tee connector is filled with water (same operations are done for valeraldehyde and HCl). When the required temperatures are reached and stable, the pumps are started one after the other according to their position in the system (P01 is switched on first). If the installation is equipped with a BPR, the pressure of the BPR is adjusted to the desired value. Once the flow rates are stable, the test is started.

### Sampling

Once the set timing is reached the cap of the end receiving flask is unscrewed, and a sampling vial is filled for 1 minute. Caution should be used as the solution is at temperature higher than its boiling point at 80° C. Once the vial is filled, the cap is put back on the flask, and the sampling vial is closed and left to cool down.

The measurement of the flow rate at the end of the process is to be carried out using the same steps, but instead of a sampling vial, a measuring cylinder is weighted beforehand, used and then re-weighed.

### Shutdown of the Installation

Once all the pumps are stopped, a new collecting flask is connected to the end of the installation. The back pressure regulator is switched off and the flasks containing Compound 2 and valeraldehyde are weighed and replaced by flasks containing acetone. Flasks of water, sodium dithionite and HCl are weighed and replaced by water flasks. The pumps are then set to their respective flow and clean-up is carried over at least 20 minutes. Inspection for leaks is carried out after 20 minutes.

### B. Experimental Apparatus and Conditions for Example 1

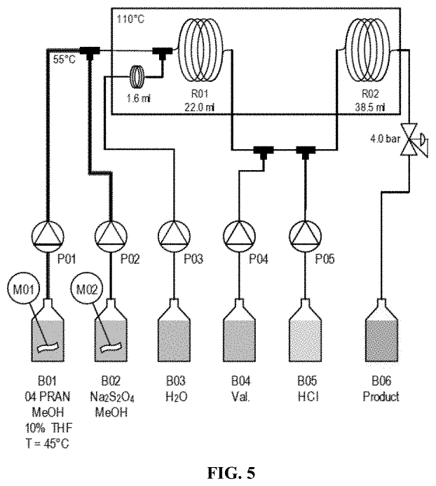

The apparatus used for the first runs of the continuous process is presented in FIG. 5. To avoid sodium dithionite plugs and to improve its dissolution inside the system, the BPR was positioned between the two reactors instead of being positioned at the end of the setup. This configuration is presented in FIG. 2.

In this configuration, the premixed flow of Compound 2 and sodium dithionite suspension [1] were mixed with preheated water (by using the heating loop [2]) in the Tee connector [3] and then introduced in a continuous reactor R01 [4]. Once reduction was achieved, the reaction mixture was mixed with hydrochloric acid [5] in the second Tee connector [6] and then passed through the BPR [7]. The BPR prevents gas formation and improves the dissolution of sodium dithionite inside continuous reactor R01 while hydrochloric acid degrades the remaining insoluble particles of sodium dithionite. After the BPR, the valeraldehyde [8] was mixed in the last Tee connector [9] with the reaction. Valeraldehyde, was added after the BPR to prevent the formation of a by-product precipitation upstream. The reaction mixture was continuously introduced in the additional continuous reactor R02 [10] and Compound 1 was collected into flask B06 upon leaving the reactor from [11]. The reaction was performed without any clogging issues, and pressures in the two peristaltic pumps (P01 and P02) were stable (4.0 to 4.3 bar). By-product precipitation was observed in the Compound 1 collection flask (B06) but no issue was observed due to tube coating. From an analytical point of view, the best results of this study were achieved with the apparatus in FIG. 2, in experiment LJ X03 94, wherein complete conversion of Compound 2 was obtained, the yield of Compound 1 was 79% with UHPLC assay of 78% (determined by UHPLC quantification of the Compound 1 formed according to the amount of Compound 2 introduced into the system).

Conditions and results from other experiments with the apparatuses previously discussed in this Example are shown in the tables below. Unless otherwise indicated, R01 is placed after the addition of water and R02 after the addition of valeraldehyde and HCl. The BPR is located at the exit of the R02.

| | 04 PRAN in MeOH | | | Na₂S₂O₄ in MeOH | | | Valeraldehyde | | HCl | |
|---|---|---|---|---|---|---|---|---|---|---|
| | Conc. | THF | | Conc. | | H₂O | Assay | | Conc. | |
| Attempt | [g/L] ¹ | [% V/V] | T [° C.] | [g/L] | T [° C.] | T [° C.] | [%] | T [° C.] | [%] | T [° C.] |
| LJ X03 83 | 15.0 | 10.0 | 45 | 31.1 | amb | amb | 97 | amb | 32 | amb |
| LJ X03 84 | 15.0 | 10.0 | 45 | 31.3 | amb | amb | 97 | amb | 32 | amb |
| LJ X03 85 | 15.0 | 10.0 | 45 | 31.3 | amb | amb | 97 | amb | 32 | amb |
| LJ X03 86 | 15.0 | 10.0 | 45 | 31.5 | amb | amb | 97 | amb | 32 | amb |
| LJ X03 87 | 15.0 | 10.0 | 45 | 31.5 | amb | amb | 97 | amb | 32 | amb |
| LJ X03 88 | 14.9 | 10.0 | 45 | 31.5 | amb | amb | 97 | amb | 32 | amb |
| LJ X03 89 | 14.9 | 10.0 | 45 | 31.5 | amb | amb | 97 | amb | 32 | amb |
| LJ X03 90 | 14.9 | 10.0 | 45 | 31.5 | amb | amb | 97 | amb | 32 | amb |
| LJ X03 91 | 14.9 | 10.0 | 45 | 31.5 | amb | amb | 97 | amb | 32 | amb |
| LJ X03 92 | 14.9 | 10.0 | 45 | 31.5 | amb | amb | 97 | amb | 32 | amb |
| LJ X03 93 | 14.9 | 10.0 | 45 | 31.4 | amb | amb | 97 | amb | 32 | amb |
| LJ X03 94 | 14.9 | 10.0 | 45 | 31.4 | amb | amb | 97 | amb | 32 | amb |

¹ Without taking into account the THF

| | 04 PRAN in MeOH | | | Na₂S₂O₄ in MeOH | | | H₂O | | | Valeraldehyde | | | HCl | | |
|---|---|---|---|---|---|---|---|---|---|---|---|---|---|---|---|
| Attempt | #¹ | F [g/min] | Pump | #¹ | F [g/min] | Pump | #¹ | F [g/min] | Pump | #¹ | F [g/min] | Pump | #¹ | F [g/min] | Pump |
| LJ X03 83 | 1 | 4.45 | P074 | 2 | 4.00 | P075 | 3 | 6.56 | P052 | 4 | 0.18 | P051 | 5 | 0.40 | P053 |
| LJ X03 84 | 1 | 4.45 | P074 | 2 | 4.00 | P075 | 3 | 6.56 | P052 | 4 | 0.18 | P051 | 5 | 0.40 | P053 |
| LJ X03 85 | 1 | 4.45 | P074 | 2 | 4.00 | P075 | 3 | 6.56 | P052 | 4 | 0.18 | P051 | 5 | 0.40 | P053 |
| LJ X03 86 | 1 | 4.45 | P074 | 2 | 4.00 | P075 | 3 | 6.56 | P052 | 4 | 0.18 | P051 | 5 | 0.40 | P053 |
| LJ X03 87 | 1 | 4.45 | P074 | 2 | 4.00 | P075 | 3 | 6.56 | P052 | 4 | 0.18 | P051 | 5 | 0.40 | P053 |
| LJ X03 88 | 1 | 4.45 | P074 | 2 | 4.00 | P075 | 3 | 6.56 | P052 | 4 | 0.18 | P051 | 5 | 0.40 | P053 |
| LJ X03 89 | 1 | 4.45 | P074 | 2 | 4.00 | P075 | 3 | 6.56 | P052 | 4 | 0.18 | P051 | 5 | 0.40 | P053 |
| LJ X03 90 | 1 | 4.45 | P074 | 2 | 4.00 | P075 | 3 | 6.56 | P052 | 4 | 0.18 | P051 | 5 | 0.40 | P053 |
| LJ X03 91 | 1 | 4.45 | P074 | 2 | 4.00 | P075 | 3 | 6.56 | P052 | 4 | 0.18 | P051 | 5 | 0.40 | P053 |
| LJ X03 92 | 1 | 4.45 | P074 | 2 | 4.00 | P075 | 3 | 6.56 | P052 | 4 | 0.18 | P051 | 5 | 0.40 | P053 |
| LJ X03 93 | 1 | 4.45 | P074 | 2 | 4.00 | P075 | 3 | 6.56 | P052 | 4 | 0.18 | P051 | 5 | 0.40 | P053 |
| LJ X03 94 | 1 | 4.45 | P074 | 2 | 4.00 | P075 | 3 | 6.56 | P052 | 4 | 0.18 | P051 | 5 | 0.40 | P053 |

¹Order of introduction into the system (First = 1).

| | Thermostatisation | | Reactor R01 | | | Reactor R02 | | | Cooling | pressure |
|---|---|---|---|---|---|---|---|---|---|---|
| Attempt | Mode ¹ | T [° C.] | V [mL] | L [cm] | T [° C.] | V [mL] | L [cm] | T [° C.] | loop | [bar] |
| LJ X03 83 | C | 55 | 22.0 | 1110 | 110 | 38.5 | 1945 | 110 | No | 4.0 |
| LJ X03 84 | C | 55 | 22.0 | 1110 | 110 | 38.5 | 1945 | 110 | No | 4.0 |
| LJ X03 85 | C | 55 | 22.0 | 1110 | 110 | 38.5 | 1945 | 110 | No | 4.0 |
| LJ X03 86 | C | 55 | 22.0 | 1110 | 110 | 38.5 | 1945 | 110 | No | 4.0 |
| LJ X03 87 | C | 55 | 22.0 | 1110 | 110 | 38.5 | 1945 | 110 | No | 2.7 |
| LJ X03 88 | C | 55 | 22.0 | 1110 | 110 | 38.5 | 1945 | 110 | No | 2.7 |
| LJ X03 89 | C | 55 | 22.0 | 1110 | 110 | 38.5 | 1945 | 110 | No | 2.7 |
| LJ X03 90 | C | 55 | 22.0 | 1110 | 110 | 38.5 | 1945 | 110 | No | 2.7 |
| LJ X03 91 | C | 55 | 22.0 | 1110 | 110 | 38.5 | 1945 | 110 | No | 4.0 |
| LJ X03 92 | C | 55 | 22.0 | 1110 | 110 | 38.5 | 1945 | 110 | No | 4.0 |
| LJ X03 93 | C | 55 | 22.0 | 1110 | 80 | 38.5 | 1945 | 80 | No | — |
| LJ X03 94 | C | 55 | 22.0 | 1110 | 80 | 38.5 | 1945 | 80 | No | 4.0 ² |

¹ A: 04 PRAN line thermostatised using the jacket (45° C.) from the flask to the first Tee connector, pump excluded.
B: A + Thermostatisation (by the jacket) between the Na₂S₂O₄ pump and the second Tee connector).
C: A + Thermostatisation (by the jacket) between the Na₂S₂O₄ pump and the inlet of the thermostatic bath containing the reactor. The water flow is heated inside the bath.
² The BPR was positioned between the 2 reactors, the HCl is added before the BPR.

| | | | | Water/ | Yield | Area |
|---|---|---|---|---|---|---|
| | Na₂S₂O₄ | HCl | Val. | dithionite | (06-PRAN) | rel 1 |
| Attempt | eq. | eq. | eq. | ratio | [%] | [%S] |
| LJ X03 83 | 3.7 | 15.0 | 8.4 | 446 | 60.0 | 74.0 |
| LJ X03 84 | 3.7 | 15.0 | 8.4 | 446 | ND | ND |
| LJ X03 85 | 3.7 | 15.0 | 8.4 | 446 | ND | ND |
| LJ X03 86 | 3.7 | 15.0 | 8.4 | 446 | ND | ND |
| LJ X03 87 | 3.7 | 15.0 | 8.4 | 446 | ND | ND |
| LJ X03 88 | 3.7 | 15.0 | 8.4 | 446 | 71.0 | 75.0 |
| LJ X03 89 | 3.7 | 15.0 | 8.4 | 446 | 82.0 | 77.0 |
| LJ X03 90 | 3.7 | 15.0 | 8.4 | 446 | 72.0 | 75.0 |
| LJ X03 91 | 3.7 | 15.0 | 8.4 | 446 | 72.0 | 74.0 |
| LJ X03 92 | 3.7 | 15.0 | 8.4 | 446 | 60.0 | 73.0 |
| LJ X03 93 | 3.7 | 15.0 | 8.4 | 446 | 65.0 | 74.0 |
| LJ X03 94 | 3.7 | 15.0 | 8.4 | 446 | 79.0 | 78.0 |

### C. Comparison Between the Batch and the Continuous Process According to Example 1

A comparison of the parameters, conditions and results of batch and continuous processes are summarized in Table 3.

TABLE 3
Comparison between the batch and the continuous process.

| | | | | | | 06-PRAN | |
|---|---|---|---|---|---|---|---|
| | | Equivalents | | Conditions | | | UHPLC |
| Process | Na₂S₂O₄ | Valeraldehyde | HCl | T [° C.] | p [bar] | Yield [%] | assay [%] ¹ |
| Batch | 3.1 | 1.2 | 2.5 | 70 | atm | 49 ² to 79 ² | 90 ³ |
| Continuous | 3.7 | 8.4 | 15.0 | 80 | 1^{st} step: 4.0 | 79 ³ | 78 ³ |
| | | | | | 2^{nd} step: atm | | |

Results of the continuous process were determined by the UHPLC at the HEIA-FR, the error of measurement was evaluated at 2%.
¹ This value corresponds to the area of Compound 1 divided by the sum of areas of all peaks.
² Isolated products.
³ Determined after the reaction without any work-up.

Equivalents used in the continuous process are all higher than the ones used in the batch process, especially for the cyclization step. For the reduction step, the equivalents of sodium dithionite should be decreased to prevent gas formation inside the reactor and to adjust the water to sodium dithionite ratio by limiting the final dilution. Regarding the second step, experiments showed that valeraldehyde and hydrochloric acid eq. cannot be drastically reduced, particularly due to the concentration of the amine derivative 1 at the R01 outlet. In the batch process, a well-known by-product (06-PRAN.i3) is formed and easily removed in the work-up by filtration. In the continuous process, another by-product with unknown structure is formed. This by-product precipitates inside the reaction mixture once cooled and can coat the tube when solvent is vaporized, clogging the setup after a few minutes. It was discovered that, by carrying out this reaction at 80° C. under atmospheric pressure, the clogging problems are eliminated.

From the analytical point of view, 90% UHPLC assay was achieved at the end of the reaction on the batch process instead of 78% for the continuous process. Since complete conversion of Compound 2 and amine derivative occurred in the continuous process, the decreased of this value is due to a lower reaction selectivity than in the batch process. Comparison with the batch reduction step (analytical composition determination) was not performed. Due to the high excess of reagents in the second step, presence of side reactions such as the formation of the unknown solid by-product is highly probable and would lead to a loss of selectivity. Moreover, the yield obtained for the batch process (49% to 79%) is for the isolated product (i.e., was determined after a purification step). For the continuous process, yield values are based on the amount of Compound 1 in the final reaction mixture (i.e. without any isolation or purification steps). Thus, the yield of the isolated or purified product in the continuous process will be less than 79%.

Comparisons of economics and productivity considerations can be found in U.S. Provisional Application No. 62/818,627, filed Mar. 14, 2019, and U.S. Provisional Application No. 62/895,349, filed Sep. 3, 2019, all of which are incorporated herein by reference.

The inventors have conceived of several possibilities for future investigation. First, the solution concentration of Compound 2 can be increased by using an efficient tube heating system, which may permit reduction of the sodium eq. and the dilution of this suspension. Other equivalents (valeraldehyde and hydrochloric acid) can be decreased, which may prevent by-products formation and improve the selectivity of the reaction. By using more powerful pumps, flows rates as well as the pressure inside the system can be raised, which may present novel process windows. Pumping the sodium dithionite suspension from top to bottom may prevent plug formation inside the feeding tube of the P02 pump.

By applying these one or more of modifications (or others), efficiency and/or productivity may improve and/or production costs may be reduced. To be implemented in a production plant, scale-up of the novel continuous process disclosed herein may be possible by applying the numbering-up strategy (using several units in parallel).

### D. Example of Continuous Synthesis of Compound 2 From Compound 1

Using the apparatus of FIG. 2, 12.2 g of Compound 2 is introduced into the flask B01 (1000 ml), then 800 ml of methanol and 80 ml of THF (10% V/V) are added. The mixture is stirred at 450 rpm and heated at 45° C. using a heated stirring plate (“PRECIS” mode is used if the plate has several heating modes). Then, the flask is connected to pump P01 (P074). The solution at 45° C. is a limpid red liquid.

The B02 flask (500 ml) is first washed with acetone and then dried. Once dry, 550 ml of methanol is then introduced into the flask and the solvent is flushed with argon (the bottle is topped with a balloon filled with argon). 20.3 g of sodium dithionite (85%) is introduced into the flask. This flask is stirred at 400 rpm by using a stirring plate. Finally, the flask is connected to pump P02 (P075). The suspension is a whitish suspension.

The B03 flask (1000 ml) is filled with demineralized water and connected to pump P03 (P053).

B02 and B03 flasks (250 ml) are filled with valeraldehyde (97%, P052) and hydrochloric acid (32%, P051) respectively. Flasks are then connected to their pump.

The pressure of the back pressure regulator is set at 4.0 bar. A thermostatic bath used for the jacket is set to 55° C. The thermostatic bath containing the reactor is set to 80° C. Flows rates are adapted to solution concentrations by respecting these three points:
Flow rate of P02 (sodium dithionite in methanol)=4.0 g/min;
Equivalent of sodium=3.7;
Ratio of water to sodium dithionite=446.

Once the set temperatures of the jacket and the reactor are reached, pumps are switched on and product is collected into 100 ml flasks. Reaction mixture produced during the 12 first minutes is not collected.

When the set temperature of the reactor is reached, pumps are switched on and product is collected into B06 (2000 ml). Reaction mixture produced during the 6 first minutes is not collected.

### Example 2

### A. Operating procedures for Example 2

### Preparation of solutions

Compound 2 is introduced into the flask B01, then methanol is added to obtain a solution of 15 g/l 10% (V/V) of THF are added to flask B01. The mixture is stirred at 400 rpm and heated at 45° C., e.g., using a heated stirring plate.

Where an apparatus comprising a cartridge is used, water is added in the B01 flask to obtain a solution where water represents 140 equivalents of Compound 2.

A B02 flask is first washed with acetone and then dried over pressurized air. Methanol is then introduced into the flask and the solvent is flushed with argon (the bottle is topped with a balloon filled with argon). Finally, sodium dithionite is introduced into the flask. Another flush can be performed. Quantities sufficient to obtain a 36 g/l suspension of sodium dithionite in methanol are used.

Water, valeraldehyde and hydrochloric acid are added to their respective flasks. Flasks are all equipped with a ventilation valve and a pumping tube, except the dithionite flask B02 which is equipped with an argon balloon instead of a ventilation valve. The B01 flask was also equipped with a thermometer.

Where an apparatus comprising a cartridge is used, the cartridge is cleaned with water and then acetone then dried over pressurised air being careful not to expel the filters from the cartridge. The sodium dithionite is then inserted in the desired mass and packed using the cartridge wheel.

### Start-Up of the Installation

The start-up of the process starts by switching on both the thermostatic baths at 45° C. for the jacket 1 and 50° C. for the reactor 1. Then all the solutions are made and linked to their respective pumps in this order: water, HCl, valeraldehyde first, then 04-PRAN. At this point, the temperature of the reactor baths is switched to 80° C. This temperature step is made to prevent the washing solvent (acetone) to be vaporised beforehand. Then the solution of dithionite is made and implemented. All the pumps are primed after the temperatures are stable. The BPR is started and then all the pumps are started in reverse order (first is 5, second is 4 . . . ).

### Sampling

Once the set timing is reached the cap of the end receiving flask is unscrewed, and a sampling vial is filled for 1 minute. Caution should be used as the solution is at temperature higher than its boiling point at 80° C. Once the vial is filled, the cap is put back on the flask, and the sampling vial is closed and left to cool down.

The measurement of the flow rate at the end of the process is to be carried out using the same steps, but instead of a sampling vial, a measuring cylinder is weighted beforehand, used and then re-weighed.

### Shutdown of the Installation

Once all the pumps are stopped, a new collecting flask is connected to the end of the installation. The back pressure regulator is switched off and the flasks containing Compound 2 and valeraldehyde are weighed and replaced by flasks containing acetone. Flasks of water, sodium dithionite and HCl are weighed and replaced by water flasks. The pumps are then set to their respective flow and clean-up is carried over at least 20 minutes. Inspection for leaks is carried out after 20 minutes.

### B. Details Regarding Apparatus for Example 2

The apparatus used for the example discussed below is similar to that depicted in FIG. 9, which was based on the apparatus used for LT X093 94 discussed above, but with inverted pumps, i.e., pumps P03 and P04 are Ismatec rotary piston pumps. P05 is a KNF membrane pump.

Reactors are made from the same tubing utilised for the different transfer, rolled around a stainless-steel cylinder and are 12-metre-long for R01 and 20 metre long for R02. The water heater consists of a tube of 2.8 metre rolled and held by two wraps. The thermostatic bath for the reactor is filled with oil. The double jacket and its thermostatic bath are filled with water.

Additional details concerning particular parts of the apparatus can be found above in Example 1 and in the tables below.

### 1 Reactor Features

| Reactor type | length [m] | tube diameter | volume [m3] | volume [ml] |
|---|---|---|---|---|
| Heater | 2.8 | 0.00158 | 5.48987E−06 | 5.48987033 |
| R01 reduction | 12 | 0.00158 | 2.3528E−05 | 23.5280157 |
| R02 cyclisation | 20 | 0.00158 | 3.92134E−05 | 39.2133595 |

| | flow rate | | volumetric | tau | Tau | Tau |
|---|---|---|---|---|---|---|
| | mass | density | flow rate | R01 | R02 | total |
| | [g/min] | [kg/m3] | [kg/s] | [min] | [min] | [min] |
| 04-PRAN | 1.9185 | 791 | 4.04E-08 | 9.70 | 16.17 | 25.87 |
| CHV-004 | | | | | | |

### C. Conversion, Yield and Selectivity for Experiment CHV-004

The conversion, yield and selectivity for this reaction are shown in Table 4.

TABLE 4
Conversion, yield and selectivity for CHV-004

| | Compound 2 | Compound 1 | Compound 2 | Compound 1 | | | |
|---|---|---|---|---|---|---|---|
| | concentration | concentration | molar conc. | molar conc. | | | |
| Conversion | [g/l] | [g/l] | [M] | [M] | conversion | yield | selectivity |
| sample 1 15 min | 1.02E+00 | 5.38E−01 | 3.19E−03 | 1.51E−03 | 7.48E+01 | 2.52E+01 | 5.41E+01 |
| sample 2 20 min | 0.00E+00 | 4.65E−01 | 0.00E+00 | 1.30E−03 | 1.00E+02 | 2.18E+01 | 2.18E+01 |
| sample 3 25 min | 1.44E+00 | 5.38E−01 | 4.49E−03 | 1.51E−03 | 7.48E+01 | 2.52E+01 | 1.02E+02 |
| sample 4 30 min | 0.00E+00 | 3.57E−01 | 0.00E+00 | 1.00E−03 | 1.00E+02 | 1.67E+01 | 1.67E+01 |
| sample 5 35 min | 0.00E+00 | 1.15E+00 | 0.00E+00 | 3.22E−03 | 1.00E+02 | 5.40E+01 | 5.40E+01 |
| sample 6 40 min | 0.00E+00 | 6.94E−01 | 0.00E+00 | 1.94E−03 | 1.00E+02 | 3.26E+01 | 3.26E+01 |
| sample 7 45 min | 0.00E+00 | 4.77E−01 | 0.00E+00 | 1.34E−03 | 1.00E+02 | 2.24E+01 | 2.24E+01 |
| sample 8 50 min | 0.00E+00 | 1.19E+00 | 0.00E+00 | 3.33E−03 | 1.00E+02 | 5.57E+01 | 5.57E+01 |
| sample 9 55 min | 0.00E+00 | 8.63E−01 | 0.00E+00 | 2.42E−03 | 1.00E+02 | 4.05E+01 | 4.05E+01 |
| sample 10 60 min | 0.00E+00 | 6.70E−01 | 0.00E+00 | 1.88E−03 | 1.00E+02 | 3.14E+01 | 3.14E+01 |

### D. Comparison of Example 2, Experiment CHV-004, and Example 1, Experiment LJ X03 94

The outflow rate of Experiment CHV-004 was two times lower than that for Experiment LJ X03 94, resulting in a longer operating time (1 hour for Experiment CHV-004 versus 17.8 mins for LJ X03 94). The increase in operating time did not impact the conversion, as Samples 4 to 10 of Experiment CHV-004 showed total conversion. However, the selectivity and yield of this experiment was lower than Experiment LJ X03 94.

Selectivity was calculated as shown in equation 1:

$$Equation 1 \text{:} Selectivity calculus$$

$$06 PRAN / 04 PRAN = \frac{v 04 PRAN}{v 06 PRAN} * \frac{final molar conc . 06 PRAN - initial molar conc . 06 PRAN}{initial molar Flux 04 PRAN - final molar conc . 04 PRAN}$$

Although this calculation does not take byproducts or amine intermediate 1 into account, it can seen that there is room for improvement in selectivity. The inventors believed that one solution could be an increase in residence time for both whole reactions.

A 26 minutes residence time was found for Experiment CHV-004.

The inventors believed that another solution could be to maximize contact between the dithionite solution and Compound 2 by using a static mixing reactor.

Yields in Experiment CHV-004 were lower than in LJ X03 94, which was 79%. Factors potentially affecting the accuracy of this yield calculation include the fact that, when the sample are taken, methanol is above its boiling point, resulting in a mass loss. Another factor is that, during the experiment of Example 2, all the samples were double-phased. No reaction lasted long enough to reach five times the residence time (2 hours 30 minutes), the landmark for the steady state for a flow reaction. Together, these led the inventors to believe that there was opportunity to obtain additional product from the bottom layer, increasing yield and selectivity, for example, by modifying the flow rate of reactant. Furthermore, it was hypothesized that the previously noted discoloration of the solution could be used as a calorimetry method, which could be developed and implemented directly at the output of the installation to refine the different flowrates according to the color of the product.

### Example 4

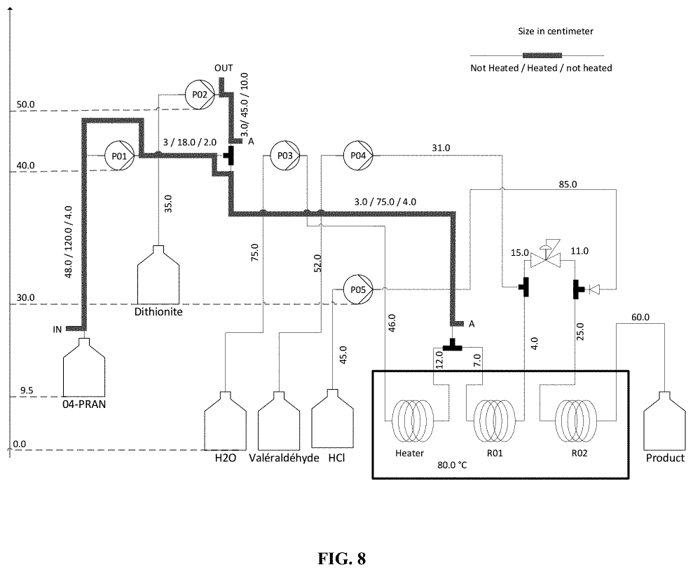

Various apparatus modifications were investigated by the inventors. The apparatus shown in FIG. 7 was adapted from that used in LT X03 94 discussed above. The apparatus shown in FIG. 8 was adapted from that shown in FIG. 7, but the apparatus in FIG. 8 was fitted with a shorter length (35 cm rather than 45 cm) of tube connecting the flask containing the dithionite solution to its pump, P02. The short tube length improved pumping of the dithionite solution, which was observed to form a precipitate in the flask. Further improvement of pumping was observed by using the strongest pump available (Prominent Gamma 4) to handle the dithionite solution and inversion of pump P01 and P02, as shown in FIG. 9. The inversion was a successful idea as a new parameter appeared: the oscillation of the dithionite solution in a Tee connector before its mixing with the Compound 2 solution. This oscillation greatly increased the mixing of the two solutions and helped to fill the end part of the tube with more solid, resulting in a better production of amine intermediate 1 and a better conversion into Compound 1.

To eliminate the use of two pumps P02 and P03, the inventors further modified the apparatus by implementing a cartridge filled with dithionite, as shown in FIG. 9. This setup revealed some new challenges, as the sodium dithionite is not soluble in MeOH. Accordingly, the inventors decided to add water in the Compound 2 solution to dissolve the dithionite in the cartridge.

### Solution Investigation

To reduce the number of flasks required in the apparatus, the inventors attempted to add water in a stoichiometric quantity in the Compound 2 solution to go through the cartridge. To make such an adjustment required knowing the effect of water in the solution. Starting with the same concentration of each product in the Compound 2 solution, different equivalents of water were added to determine whether there exists a limit to the amount of water in the solution, and if so, to determine that. Results of that investigation are shown in Table 5.

TABLE 5
Equivalents tested for the crystallisation
of Compound 2 with water

| Equivalent of water | |
|---|---|
| in the Compound 2 | Crystallisation |
| solution | observed? |
| 50 | No |
| 100 | No |
| 150 | No |
| 200 | No |
| 214 | No |
| 400 | Slightly |
| 1300 | yes |

These experiments were performed at 45° C., which is the temperature of the solution before use in the continuous process.

214 equivalents represents a theoretical concentration of 35.95 gram per litre using the 220 gram per litre solubility in water given in the literature. In order to keep a safety margin at 25° C., the temperature at which the solution is prepared, 140 equivalents were used in all the cartridge experiments, to avoid any crystallisation if a temperature loss appeared in the cartridge and to ensure that no degradation due to an excess of water in the cartridge could occur.

Effective solubility of dithionite was calculated by drying the remaining solid in the cartridge and weighing it before and after the experiment. A brief hypothetical verification was performed beforehand to confirm that two potential problems were avoided. First, it was verified that the cleaning of the cartridge with acetone did not affect its mass. In other words, it was shown that sodium dithionite is not soluble in acetone. Second, it was confirmed that the remaining solid in the cartridge contained only sodium dithionite and no residual Compound 2 and that there is no crystallisation due to a temperature loss in the cartridge.

### Handling of Sodium Dithionite

Handling of the sodium dithionite in the continuous process presented several hurdles during this project. For example, sodium dithionite is not stable in water. Thus, solid sodium dithionite should not contact the water before mixing with Compound 2, such that, after mixing, the water solubilises the dithionite in the Compound 2 solution and reacts with Compound 2 to reduce its nitro group into an amino group. As previously discussed, issues of pumping the dithionite were observed, thus various changes to the apparatus were investigated to ensure the correct amount of dithionite was delivered into the continuous process. Another factor that was explored was the total concentration of dithionite in the starting solution. Tests were performed to determine if, by speeding up the mixing in the solution or increasing the concentration of the solution a bigger part of solid dithionite would be pumped. Investigations including doubling the concentration of solid dithionite to determine whether the total solid mass pumped in one minute would increase. Also investigated was whether the mixing rate was increased to 1000 rpm would achieve the same goal. Finally, a solution filled with the minimum liquid to be pumped without agitation was tested. A control solution with the standard parameter was tested at the end to be able to compare. The summary of the experiments in Table 6.

TABLE 6
Experiments involving dithionite slurry

| Concentration of | | Mass pumped |
|---|---|---|
| dithionite [g/l] | Agitation [rpm] | in one minute [g] |
| 100 | 500 | 0.214 |
| Paste of dithionite | — | 0.023 |
| 72 | 500 | 0.237 |
| 72 | 1000 | 0.233 |
| 36 | 500 | 0.232 |

The maximum was achieved by a using a 72 g/l solution of dithionite with stirring at 500 rpm and a 0.237 gram pumped in one minute. In view of the small increase in pumpability, a different approach was sought. This involved changing the main motor of the apparatus as previously discussed. The inventors determined that pump inversion and using a correct amount of dithionite stacked before the Tee connector before the mixing with Compound 2 and an oscillatory movement going back and forth as previously shown.

### Packed Bed Reactor

The final investigations performed by the inventors pertained to removing from the continuous process the use of dithionite slurry solution and the addition of water. The result was the implementation of a solid-containing cartridge into the apparatus as shown in FIG. 9.

During the investigations, two of the issues encountered were a vaporisation of the reactional milieu in R01 and poor dissolution of dithionite in the cartridge. First, vaporisation of the milieu in the reactor occurred in experiments CHV-006 and CHV-008, discussed below, which diminished the residence time for both the steps of the synthetic reaction. Vaporisation was determined to be due to loss of pressure inside the cartridge and another pressure leak. Doubling the flow rate of Compound 2 in the cartridge, as in experiment CHV-007, eliminated vaporisation.

Second, to address the lack of solubilisation of dithionite in the cartridge, the inventors attempted to use a five-time greater amount of dithionite in the cartridge, to try to increase the mass transfer inside the packed bed. This attempt proved that the limiting factor is not the contact time between the solid and the solution but rather the dithionite/water ratio. It was also determined that, even with a smaller ratio, the experiment CHV-008 and the large amount of dithionite inside the cartridge led to an increase in solubility (although still below that reported in the literature). Results of these experiments are set forth below in Tables 7, 8, and 9.

TABLE 7
Summary for Experiment CHV-006

| | total flow | | | | |
|---|---|---|---|---|---|
| nominal flow product | Compound 2 | | | Volume engaged | Dissolved |
| CHV-006 | [g/min] | Equiv. | [mmol/min] | [ml] | mass [g] |
| | 2.060 | | — | | |
| Compound 2 | 0.003 | 1.000 | 0.009 | | |
| THF | 0.079 | 26.390 | 1.091 | | |
| MeOH | 1.561 | 523.650 | 48.725 | | |
| H₂O | 0.417 | 140.000 | 23.162 | 25.043 | |
| Equiv. total | | 691.040 | | | |
| Dithionite | 0.030 | 18.327 | 0.170 | | 1.776 |
| Solubility of | 70.919 | | | | |
| dithionite | | | | | |

TABLE 8
Summary for Experiment CHV-007

| nominal flow | flow total | | | Volume | Dissolved |
|---|---|---|---|---|---|
| product | Compound | | [mmol/ | engaged | mass |
| CHV-007 | 2 [g/min] | Equiv. | min] | [ml] | [g] |
| | 4.779 | | — | | |
| Compound 2 | 0.007 | 1.000 | 0.022 | | |
| THF | 0.182 | 26.380 | 2.530 | | |
| MeOH | 3.622 | 523.660 | 113.039 | | |
| H₂O | 0.968 | 140.000 | 53.734 | 67.780 | |
| Equiv. total | | 691.040 | | | |
| Dithionite | 0.029 | 7.770 | 0.167 | | 2.038 |
| Solubility of | 30.068 | | | | |
| dithionite | | | | | |

TABLE 9
Summary for Experiment CHV-008

| nominal flow | flow total | | | Volume | Dissolved |
|---|---|---|---|---|---|
| product CHV- | Compound | | [mmol/ | engaged | mass |
| 008 | 2 [g/min] | Equiv. | min] | [ml] | [g] |
| | 5.118 | | — | | |
| Compound 2 | 0.007 | 1.000 | 0.023 | | |
| THF | 0.195 | 26.380 | 2.709 | | |
| MeOH | 3.878 | 523.660 | 121.043 | | |
| H₂O | 1.037 | 140.000 | 57.538 | 48.000 | |
| Equiv. total | | 691.040 | | | |
| Dithionite | 0.058 | 14.352 | 0.331 | | 3.455 |
| Solubility of | 71.979 | | | | |
| dithionite | | | | | |

1. A continuous process comprising reacting Compound 2 to reduce its nitro group and cyclizing the product with valeraldehyde to produce Compound 1,
wherein a composition comprising Compound 2 and optionally at least one solvent is continuously passed through a packed bed reactor comprising sodium dithionite,
wherein the resulting composition is continuously transferred to a continuous process reactor,
wherein the composition comprising the reaction product of sodium dithionite and Compound 2 is combined with valeraldehyde and passed through a back pressure regulator,
wherein hydrochloric acid is combined with the composition comprising the reaction product of sodium dithionite, Compound 2, and valeraldehyde, and
wherein the resulting composition is continuously transferred to an additional continuous process reactor, and
wherein Compound 1 is continuously produced.

2. The continuous process according to claim 1, wherein the continuous process reactor and the additional continuous process reactor are in a thermostatic bath.

3. The continuous process according to claim 2, wherein the thermostatic bath is heated to 80° C.

4. The continuous process according to claim 1, wherein the composition comprising Compound 2 further comprises water.

5. The continuous process according to claim 1, wherein composition comprising Compound 2 is heated.

6. The continuous process according to claim 1, wherein the composition comprising sodium dithionite is heated.

7. The continuous process according to claim 1, wherein the apparatus used for the continuous process is substantially the same as shown in FIG. 9.

8. A continuous process comprising reacting Compound 2 to reduce its nitro group and cyclizing the product with valeraldehyde to produce Compound 1,
wherein a composition comprising Compound 2 and optionally at least one solvent is continuously combined with a composition comprising sodium dithionite and optionally at least one solvent,
wherein the composition comprising the reaction product of sodium dithionite and Compound 2 is combined with water,
wherein the resulting composition is continuously transferred to a continuous process reactor,
wherein the composition comprising the reaction product of sodium dithionite, Compound 2, and water is combined with valeraldehyde and passed through a back pressure regulator,
wherein hydrochloric acid is combined with the composition comprising the reaction product of sodium dithionite, Compound 2, and valeraldehyde, and
wherein the resulting composition is continuously transferred to an additional continuous process reactor, and
wherein Compound 1 is continuously produced.

9. The continuous process according to claim 8, wherein the continuous process reactor and the additional continuous process reactor are in a thermostatic bath.

10. The continuous process according to claim 9, wherein the thermostatic bath is heated to 80° C.

11. The continuous process according to claim 9, wherein the thermostatic bath further comprises a heater through which water continuously passes.

12. The continuous process according to claim 8, wherein the composition comprising Compound 2 is heated.

13. The continuous process according to claim 8, wherein the composition the composition comprising sodium dithionite is heated.

14. The continuous process according to claim 8, wherein the water combined with the composition comprising the reaction product of sodium dithionite and Compound 2 is heated.

15. The continuous process according to claim 14, wherein the water is heated to 80° C.

16. The continuous process according to claim 8, wherein the apparatus used for the continuous process is substantially the same as shown in at least one Figure chosen from FIGS. 7, 8, and 9.

17. A continuous process reacting Compound 2 to reduce its nitro group and cyclizing the product with valeraldehyde to produce Compound 1,
wherein a composition comprising Compound 2 and optionally at least one solvent is continuously transferred to a continuous process reactor,
wherein sodium dithionite is continuously combined with the composition comprising Compound 2,
wherein the composition comprising the reaction product of sodium dithionite and Compound 2 is combined with hydrochloric acid,
wherein the composition comprising the reaction product of sodium dithionite and Compound 2 is combined with hydrochloric acid, followed by passage through a back pressure regulator,
wherein valeraldehyde is combined with the composition comprising the reaction product of sodium dithionite, Compound 2, and hydrochloric acid, and the resulting composition is continuously transferred to an additional continuous process reactor, and
wherein Compound 1 is continuously produced.

18. The continuous process according to claim 17, wherein the continuous process reactor and the additional continuous process reactor are in a thermostatic bath.

19. The continuous process according to claim 18, wherein the thermostatic bath is heated to 80° C.

20. The continuous process according to claim 18, wherein the thermostatic bath further comprises a heater through which water continuously passes.

21. The continuous process according to claim 17, wherein the pressure of the continuous process reactor is 4.0 bar.

22. The continuous process according to claim 17, wherein the pressure of the additional continuous process reactor is atmospheric pressure.

23. The continuous process according to claim 17, wherein the apparatus used for the continuous process is substantially the same as shown in at least one Figure chosen from FIGS. 2 and 3.

24. A continuous process reacting Compound 2 to reduce its nitro group and cyclizing the product with valeraldehyde to produce Compound 1,
wherein a composition comprising Compound 2 and optionally at least one solvent is continuously combined with a composition comprising sodium dithionite and optionally at least one solvent,
wherein water is added to the composition comprising the reaction product of sodium dithionite and Compound 2,
wherein the composition comprising water and the reaction product of sodium dithionite and Compound 2 is continuously transferred to a continuous process reactor,
wherein the composition comprising water and the reaction product of sodium dithionite and Compound 2 is combined with valeraldehyde,
wherein hydrochloric acid is combined with the composition comprising the reaction product of sodium dithionite, Compound 2, and valeraldehyde,
wherein the resulting composition is continuously transferred to an additional continuous process reactor, and
wherein Compound 1 is continuously produced.

25. The continuous process according to claim 24, wherein the composition comprising Compound 2 further comprises at least one solvent.

26. The continuous process according to claim 24, wherein the at least one solvent is chosen from methanol, tetrahydrofuran, and water.

27. The continuous process according to claim 24, wherein the at least one solvent is methanol and 10% tetrahydrofuran.

28. continuous process according to claim 24, wherein the composition comprising Compound 2 further comprising at least one solvent is heated to 45° C.

29. The continuous process according to claim 24, wherein the composition comprising sodium dithionite further comprises at least one solvent.

30. The continuous process according to claim 24, wherein the at least one solvent is chosen from methanol and water.

31. The continuous process according to claim 24, wherein the at least one solvent is methanol.

32. The continuous process according to claim 24, wherein the composition comprising Compound 2 and optionally at least one solvent is heated.

33. The continuous process according to claim 24, wherein the composition comprising Compound 2 and optionally at least one solvent is heated to 55° C.

34. The continuous process according to claim 24, wherein the composition comprising sodium dithionite and optionally at least one solvent is heated.

35. The continuous process according to claim 24, wherein the continuous process reactor and the additional continuous process reactor are in a thermostatic bath.

36. The continuous process according to claim 35, wherein the thermostatic bath is heated to 80° C.

37. The continuous process according to claim 35, wherein the thermostatic bath further comprises a heater through which water continuously passes.

38. The continuous process according to claim 24, wherein, after passing through the additional continuous process reactor, the composition is continuously passed through a cooling loop and/or through a back pressure regulator.

39. The continuous process according to claim 24, wherein the apparatus used for the continuous process is substantially the same as shown in at least one Figure chosen from FIGS. 4, 5, and 6.
